(19) United States
(12) Patent Application Publication
(10) Pub. No.: US 2020/0009294 A1
(43) Pub. Date: Jan. 9, 2020

(54) PROCESS FOR INSTANT NANOPOROUS BIOARTIFICIAL BONE TISSUE COMPOSITE ENGINEERING

(71) Applicants: Pali NAZIR, Jerusalem (IL); Rama NAZIR, Jerusalem (IL)

(72) Inventors: Pali NAZIR, Jerusalem (IL); Rama NAZIR, Jerusalem (IL)

(21) Appl. No.: 16/483,420

(22) PCT Filed: Jan. 29, 2018
(86) PCT No.: PCT/IL2018/050096

§ 371 Date: Aug. 4, 2019

(30) Foreign Application Priority Data
Mar. 1, 2017 (IL) 250881

(51) Int. Cl. A61L 27/36; A61L 27/18; A61L 27/46; A61L 27/56; A61L 27/58; A61L 27/12; A61L 27/54

(52) U.S. Cl. CPC A61L 27/365; A61L 27/3691; A61L 27/18; A61L 27/46; A61L 2400/12; A61L 27/58; A61L 27/12; A61L 27/54; A61L 2400/06; A61L 27/56

(57) ABSTRACT

Process for engineering instant nanoporous bio-artificial bone tissue composite is designed aiming the prevention of body immune reaction when integrated with host bone tissue and also reduces its period of integration. This nanoporous biocomposite is compounded by a number of ingredients. Hydroxylapatite (HA), or/and alpha- or beta-tricalcium phosphate (α- or β-TCP). Polycaprolactone (PCL) or polylactic acid (PLA). Any gelatinable solution or gel, then water is added in relevant quantity for producing gel or injectable solution of this biocomposite. Any substance composed of any connective collagen tissue. Recombinant Human Vascular Endothelial Growth Factor. Any kind of acrylate when the hardening and final process of all this compound is acquired immediately by ultrasound treatment.

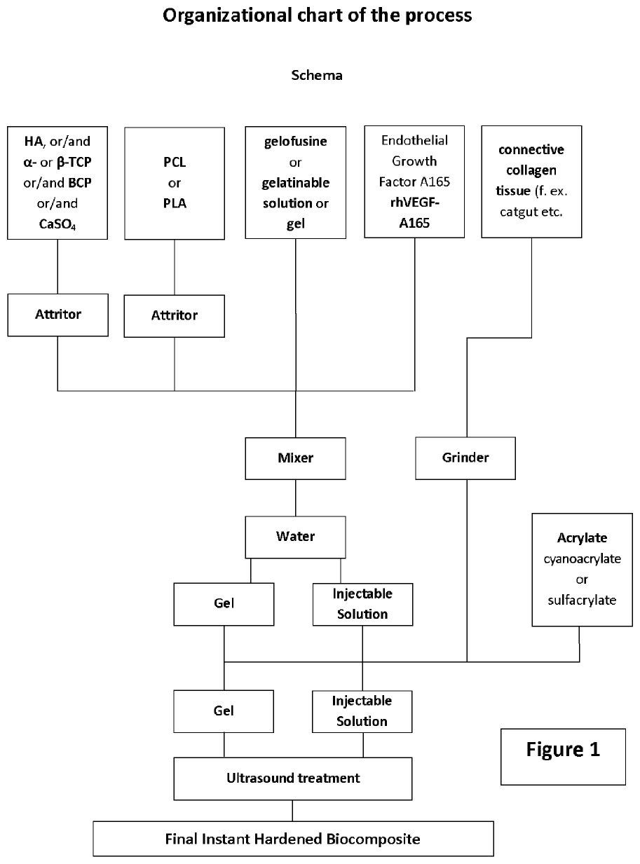

This process for engineering instant nanoporous bio-artificial bone tissue composite is designed aiming the prevention of body immune reaction when integrated with host bone tissue and also reduces its period of integration. Its composition permits instant transplantation of any implantable devices into this biocomposite because of its instant hardening property and instant integration in any part of the skeleton. This nanoporous biocomposite is compounded as follow: With first ingredient as Hydroxylapatite (HA), or/and alpha- or beta-tricalcium phosphate (α- or β-TCP), or/and biphasic calcium phosphate (BCP) which results of combining HA and TCP, or/and Calcium sulfate (CaSO₄) whose particles do not exceed 99 nm. Next ingredient, polycaprolactone (PCL) or polylactic acid (PLA) also with particles do not exceeding 99 nm. Other ingredients are: gelofusine or any gelatinable solution or gel, then water is added in relevant quantity for producing gel or injectable solution of this biocomposite. Later on, any substance composed of any connective collagen tissue (f. ex. catgut etc.). after this, recombinant Human Vascular Endothelial Growth Factor A165 (rhVEGF-A165) with a dosage inferior to 1-3 ng/ml of the final compound measured by the dose-dependant stimulation of the proliferation of Human Vascular Endothelial Cell (HUVEC). Afterwards, any kind of acrylate (cyanoacrylate or sulfacrylate etc.) used as molecular ligand, when the hardening and final process of all this compound is acquired immediately by ultrasound treatment at a frequency between 24 and 32 Khz applied for a very short period of time under 60 s. The proportions of the different components are very variable and may be adapted to the type and form of the treated bone individually. In any case the first ingredient of the compound (HA or α- or (β-TCP and so on) proportion with PCL or/and PLA is between 58% and 66% of the resulting intermediate compound. The acrylate (cyanoacrylate or sulfacrylate etc.) is never less than 2 drops and not more than 6 drops for 0.6 ml of the composite. Using 3D CAD modelling and press forming, it is possible to build with this biocomposite any part of the skeleton (human or animal). (3-TCP may be reinforced with biodegradable Fe—Mg metal phase or Fe—Ag nanocomposites. In any kind of jointure parts relevant coating (e.g. Titanium Nitrite also called Tinite (TIN) coating or Diamond Like Carbon (DLC) coating etc.) will be added. In the vertebral parts it is possible to add to this biocomposite biocompatible phospholipids.

### REFERENCES 1. Haller A. Experimentorum de ossiem formation., In Opera minora. 1763, Francisci Grasset: Lausanne; 1763:400. 2. Trueta J. The role of the vessels in osteogenesis. J Bone Joint Surg 1963; 45B:402-418. 3. Use of Sulfacrylate adhesive in gastrointestinal surgery. Savel'ev VS, Visaitov BA, Stupin IV, Sapelkina IM. Khirurgiia (Mosk). 1982 Oct;(10):89-93. 4. Ravaglioli, A. and A. Krajewski, ed. Bioceramics and the Human Body. Elsevier Applied Science, 1992. 5. Hench, Larry and June Wilson. An Introduction to Bioceramics. World Scientific Publishing Col. Ltd., 1993. 6. Fischman, Gary and Clare, Alexis, ed. “Bioceramics: Materials and Applications.” In Ceramic Transactions. The American Ceramic Society, 1995. 7. Vascular endothelial growth factor stimulates bone repair by promoting argiogenesis and bone turnover. John Street, Min Bao, Leo de Guzman, Stuart Bunting, Franklin V. Peale, Jr., Napoleone Ferrara, Hope Steinmetz, John Hoeffel, Jeffrey L. Cleland, Ann Daugherty, Nicholas van Bruggen, H. Paul Redmond, Richard A. D. Carano, and Ellen H. Filvaroff. Proceedings of the National Academy of Sciences 99(15):9656-61 August 2002. 8. VEGF and bone. E. H. Filvaroff. Department of Molecular Oncology, Genentech, Inc., South San Francisco, Calif., USA. Musculoskel Neuron Interact 2003; 3(4):304-307. 9. Vascular Endothelial Growth Factor and Angiogenesis. Ann Hoeben, Bart Landuyt, Martin S. Highley, Hans Wildiers, Allan T. Van Oosterom, and Ernst A. De Brujin. Pharmacological Reviews December 2004, 56 (4) 549-580 10. Composite Scaffolds for Bone Tissue Engineering. Min Wang. American Journal of Biochemistry and Biotechnology 2 (2): 80-84, 2006 11. Chitosan Composites for Bone Tissue Engineering —An Overview. Jayachandran Venkatesan and Se-Kwon Kim. Marine Drugs 2010, 8, 2252-2266. 12. Bernstein, M., Makarov. C., Gotman, I., Phadke, A., Radin, S., Ducheyme, P., Gutmanas, E. Y. , 2010. Low temperature fabrication of β-TCP-PCL nanocomposites for bone implants. Adv. Eng. Mater. 12, B341-B347. 13. Biocompatible β-chitin Hydrogel/Nanobioactive Glass Ceramic Nanocomposite for Periodontal Bone Regeneration. S. Sowmya, P. T. Sudeesh Kumar, K. P. Chennazhi, S. V. Nair, H. Tamura, R. Jayakumar. Trends Biomater. Artif. Organs, 25(1), 1-11 (2011). 14. The role of vascular endothelial growth factor in ossification. Yan-Qi Yang, Ying-Ying Tan, and A Bark M Rabie. International Journal of Oral Science. 2012 June; 4(2): 64-68. 15. Strong bioresorbable Ca phosphate-PLA nanocomposites with strong phase distribution by attrition milling and high pressure consolidation. Artcom Rakovsky, Irena Gotman, Eugen Rabkin, Elazar Y. Gutmanas. Journal of the Mechanical Behavior of Biomedical Materials 18 (2013) 37-46. 16. Effects of Vascular Endothelial Growth Factor 165 on Bone Tissue Engineering. Lin Feng, Hao Wu, Lingling E, Donsheng Wang. Fukui Feng, Yuwan Dong, Hongchen Liu, Lili Wang. PLOS one Dec. 20, 2013. 17. β-TCP-polyactide composite scaffolds with high strength and enhanced permeability prepared by modified salt leaching method. Artoum Rakovsky, Irena Gotman, Eugen Rabkin, Elazar Y. Gutmanas. Journal of the Mechanical Behavior of Biomedical Materials 32 (2014) 89-98. 18. Nanohydroxyapatrte-reinforced chitosan composite hydrogel for bone tissue repair in vitro and in vivo. S Dhivya, S Saravanan, T P Sastry and N Selvamurugan. Journal of Nanobiotechnology 12 Jun. 2015. 19. Microstructure, mechanical characteristics and cell compatibility of (β-tricalcium phosphate reinforced with biodegradable Fe-Mg metal phase. Sanjaya K. Swain, Irena Gotman, Ronald Unger, James Kirkpatrick, Elazar Y. Gutmanas. Journal of the Mechanical Behavior of Biomedical Materials 53 (2016) 434-444. 20. The roles of vascular endothelial growth factor in bone repair and regeneration. Kai Hu, Bjorn R. Olsen. Bone October 2016, vol.91:30-38. 21. U.S. Pat. No. 4,699,788.

1. A process for instant nanoporous bioartificial bone tissue composite engineering, the process comprising:
mixing a plurality of ingredients in an order to get a mixture, and the mixture comprising:
a first ingredient granules comprising Hydroxylapatite (HA), or/and alpha- or bioresorbable beta-tricalcium phosphate polymer (α- or (β-TCP), or/and biphasic calcium phosphate (BCP) and Fe—Ag nanocomposites;
polycaprolactone (PCL) or polylactic acid (PLA) granules;
gelofusine or any gelatinable solution or gel;
connective collagen tissue;
Recombinant Human Vascular Endothelial Growth Factor, acrylate
water in relevant quantity
wherein size of the first ingredient granules is reduced by enabling an approved process with suitable cooling measures (f. ex. high energy milling attritor etc.).

2. (canceled)

3. The process as claimed in claim 1, wherein size of the PCL or PLA granules is reduced by enabling and approved process for this procedure with suitable cooling measures (e.g. high energy milling attritor etc.).

4. The process as claimed in claim 1, wherein The association of the first ingredient granules and PCL or/and PLA is possible with a laboratory mixer adequate for the process.

5. The process as claimed in claim 1, wherein the nanoporous biocomposite with a human osteoblasts monocultures is biocompatible for the growth and survival of both cell types and cell exhibited tissue-specific markers for bone formation and angiogenesis respectively.

6. (canceled)

7. The process as claimed in claim 1, wherein the addition of Gelofusine or any gelatinable solution or gel till the transformation of the whole compound into a gel with an ad hoc addition of water to obtain an usable gel.

8. The process as claimed in claim 1, wherein the water is added to the mixture till transformation for producing gel or an injectable solution of the biocomposite.

9. The process as claimed in claim 1, wherein the grinding of any element containing connective collagen tissue (f. ex. catgut etc.) until obtaining tiniest particles as possible.

10. The process as claimed in claim 1, wherein the hardening of an element containing connective collagen tissue and the acrylate drops are attached to the compound close to the surgical procedure which is finalized with the ultrasound treatment.

11. The process as claimed in claim 1, wherein it is easy to prepare artificial bones for plastic, traumatologic and orthopedic surgeries from this composite.

12. The process as claimed in claim 1, wherein the transplants and grafts made from this instant biocomposite are free of an immune reaction, while others may have many reactions and influences on the recipient's body, those based on animal bones.

13. The process as claimed in claim 1, wherein it is possible to get transplants and build any part of skeleton (human or animal) with 3D CAD modelling and press forming made of this composite with natural bone aspect.

14. The process as claimed in claim 1, wherein TiN (Tinite) or DLC (diamond like carbon) coating or other carbon like coating onto the transplant grants it natural like friction.

15. The process as claimed in claim 1, wherein the Recombinant human Vascular Endothelial Growth Factor A165 (rhVEG F-A165) is provided and acts as a potent and effective factor for micro-vascular perfusion enhancement and for the development of new micro-vascular capillaries in an organized structural network in living tissues.

16. The process as claimed in claim 1, wherein the resulting transplant is equivalent mechanically to natural bones in hardness, torsion and flexion.
